(19) United States
(12) Patent Application Publication
(10) Pub. No.: US 2007/0219378 A1
(43) Pub. Date: Sep. 20, 2007

(54) Method for Producing Sodium Fosphenytoin

(76) Inventors: Hartmut Zinser, Schaffhausen (CH); Guisep Derungs, Zurich (CH)

Correspondence Address: HAMMER & HANF, PC, 3125 SPRINGBANK LANE, SUITE G, CHARLOTTE, NC, 28226, US

(21) Appl. No.: 10/587,189

(22) PCT Filed: Feb. 5, 2004
(86) PCT No.: PCT/CH04/00063

§ 371 Date: Apr. 3, 2007

(51) Int. Cl. C07D 233/00

(52) U.S. Cl. 548/319.1

(57) ABSTRACT

Sodium fosphenytoin, 5,5-diphenyl-3-[(phosphonooxy)methyl]imidazolidine-2,4-dione disodium salt, a known anticonvulsive, antiepileptic and antiarrhythmic, can be prepared by reacting 3-hydroxymethyl-5,5-diphenylimidazoline-2,4-dione with a phosphorous acid diester or triester activated by an oxidizing agent, whose ester groups can be selectively cleaved from the reaction product, cleaving the ester groups from the resulting phosphoric acid diester 2,5-dioxo-4,4-diphenyl-imidazolidin-1-ylmethyl ester and converting the resulting 5,5-diphenyl-3-[(phosphonooxy)methyl]imidazolidine-2,4-dione to its disodium salt. The oxidizing agent is advantageously a halogenating agent such as elemental bromine or N-bromosuccinimide, and the phosphorous acid ester used is advantageously dibenzyl phosphite. Of particular importance—even taken on its own—is the fact that a phosphoric acid diester 2,5-dioxo-4,4-diphenylimidazolidin-1-ylmethyl ester, whose phosphoric acid diester structural element can be selectively cleaved, can be converted to 5,5-diphenyl-3-[(phosphonooxy)methyl]imidazolidine-2,4-dione and the latter converted to its disodium salt in a single operation.

Sodium fosphenytoin is the abbreviated name for 5,5-diphenyl-3-[(phosphonooxy)-methyl]imidazolidine-2,4-dione disodium salt, which is used as an anticonvulsive, antiepileptic and antiarrhythmic. Preparations containing sodium fosphenytoin are marketed under the name of Cerebyx.

According to the original literature synthesis (J. Phar. Sci. 1948, 73(8), 1068-1073), sodium fosphenytoin is prepared by converting hydroxymethylphenytoin, i.e. 3-hydroxymethyl-5,5-diphenylimidazoline-2,4-dione, to 3-chloromethylphenytoin, reacting this with silver dibenzylphosphonate, cleaving the two benzyl groups in the resulting diester by catalytic hydrogenation and, finally, forming the desired disodium salt by means of sodium hydroxide solution. According to EP 0 900 227 B1, diesters of said type can be prepared, without needing to use a silver salt, by reacting 3-chloromethylphenytoin or 3-bromomethylphenytoin with an alkali metal phosphonate such as potassium or sodium dibenzylphosphonate.

It has now been found that sodium fosphenytoin can advantageously be prepared by reacting 3-hydroxymethyl-5,5-diphenylimidazoline-2,4-dione with a phosphorous acid diester or triester activated by an oxidizing agent, whose ester groups can be selectively cleaved from the reaction product, cleaving the ester groups from the resulting phosphoric acid diester 2,5-dioxo-4,4-diphenyl-imidazolidin-1-ylmethyl ester and converting the resulting 5,5-diphenyl-3-[(phosphonooxy)methyl]imidazolidine-2,4-dione to its disodium salt.

The oxidizing agent which is advantageously used to activate the phosphorous acid ester is a halogenating agent such as elemental bromine, N-bromosuccinimide, 1,3-dibromo-5,5-dimethylhydantoin, carbon tetrabromide, trichlorobromomethane, elemental chlorine, N-chlorosuccinimide, trichloroisocyanuric acid, hexachloro-acetone or the like, elemental bromine being preferred. Such a halogenation is advantageously carried out in the presence of a base, e.g. in the presence of pyridine, 2,6-lutidine, 2,4,6-collidine, triethylamine or the like. The solvent which is advantageously used is a polar aprotic solvent such as dichloromethane, acetonitrile, tetrahydrofuran, dimethylformamide, dimethylacetamide, 1-methyl-2-pyrrolidone, 1,3-dimethyl-2-imidazolidinone or the like. It is advantageous to use 0.5-5.0 equivalents, preferably 1.1-1.4 equivalents, of phosphite ester, 0.5-10.0 equivalents, preferably 1.5-3.0 equivalents, of oxidizing agent and 0.5-10.0 equivalents, preferably 1.5-3.0 equivalents, of base, based on the hydroxymethylphenytoin.

Particularly suitable ester groups which can be selectively cleaved from the reaction product are those whose cleavage can be cleaved under mild acidic conditions (e.g. tert-butyl or 2,2,2-trichloroethyl), oxidatively (e.g. silylated alkyl groups), under mild basic conditions (e.g. ethyl) or photochemically (e.g. nitrobenzyl), but especially groups which can be cleaved by hydrogenolysis, such as the benzyl group and substituted benzyl groups like 4-methoxybenzyl, 4-bromobenzyl, 2-methoxybenzyl, 2,4-dimethoxybenzyl, etc. The ester groups are advantageously identical. Phosphorous acid esters whose ester groups can be selectively cleaved from the reaction product are especially ditert-butyl phosphite, dibenzyl phosphite, bis-4-methoxybenzyl phosphite, bis-4-bromobenzyl phosphite, bis-4-nitrobenzyl phosphite, bis(2,4-dimethoxybenzyl) phosphite, bis-2,2,2-trichloroethyl phosphite, bis(2-trimethylsilylethyl) phosphite, triallyl phosphite or tribenzyl phosphite, as well as dimethyl phosphite, diethyl phosphite, trimethyl phosphite or triethyl phosphite.

The phosphorous acid esters used in the process according to the invention are known or are easily accessible by processes familiar to all those skilled in the art.

The cleavage of the ester groups from the phosphoric acid diester 2,5-dioxo-4,4-diphenylimidazolidin-1-ylmethyl ester, especially cleavage by hydrogenation, advantageously takes place in a mixture of water and a water-miscible solvent, e.g. in methanol/water, isopropanol/water, acetone/water, 2-butanone/water or the like; a buffer, e.g. acetic acid/acetate, is preferably added as well.

In one preferred embodiment of the process according to the invention, 3-hydroxymethyl-5,5-diphenylimidazoline-2,4-dione is reacted with dibenzyl phosphite activated by elemental bromine or N-bromosuccinimide, the resulting phosphoric acid 2,5-dioxo-4,4-diphenylimidazolidin-1-ylmethyl dibenzyl ester is hydrogenated, advantageously in the presence of palladium-on-active charcoal, and the resulting 5,5-diphenyl-3-[(phosphonooxy)methyl]imidazolidine-2,4-dione is converted to its disodium salt with sodium carbonate. In one particularly preferred embodiment of the process according to the invention, on the one hand the activation of the dibenzyl phosphite with elemental bromine or N-bromosuccinimide and the reaction of the activation product with 3-hydroxymethyl-5,5-diphenylimidazoline-2,4-dione, and on the other hand the hydrogenation of the phosphoric acid 2,5-dioxo-4,4-diphenylimidazolidin-1-ylmethyl dibenzyl ester and the conversion of the 5,5-diphenyl-3-[(phosphonooxy)methyl]imidazolidine-2,4-dione to its disodium salt, are each carried out in one operation. Here, the activation of the dibenzyl phosphite with elemental bromine or N-bromosuccinimide and the reaction of the activation product with the 3-hydroxymethyl-5,5-diphenylimidazoline-2,4-dione take place in particular in a mixture of acetonitrile and pyridine, and the hydrogenation of the phosphoric acid 2,5-dioxo-4,4-diphenylimidazolidin-1-ylmethyl dibenzyl ester and the conversion of the 5,5-diphenyl-3-[(phosphonooxy)methyl]imidazolidine-2,4-dione to its disodium salt take place in particular in a mixture of methanol, water and an acetic acid/sodium acetate buffer.

The temperature for the various operations of the process according to the invention is advantageously in the range from about −30° C. to about +80° C., preferably in the range from about −10° C. to about +30° C.

In contrast to the known processes discussed at the outset, the process according to the invention is carried out using not a phosphate (phosphorus in oxidation state +V) but a phosphite (phosphorus in oxidation state +III). This phosphite is activated in situ with an oxidizing agent, preferably a halogenating agent, and then reacted directly with hydroxymethylphenytoin, which is known from the literature and is readily accessible from commercially available phenytoin. In contrast to the known processes mentioned, activation of the hydroxymethylphenytoin, e.g. by conversion to the corresponding chloride or bromide, is not necessary here.

Furthermore, in the process according to the invention, especially in its preferred embodiment illustrated above, the conversion of hydroxymethylphenytoin to sodium fosphenytoin requires only two operations, whereas the known processes discussed at the outset require four operations, i.e. firstly activation of the hydroxy-methylphenytoin, secondly coupling, thirdly cleavage of the ester groups and fourthly salt formation. The number of solvents to be used in the process according to the invention is thus smaller than in the known processes mentioned. Of particular importance in this context is the second step of the process according to the invention, in which, to prepare 5,5-diphenyl-3-[(phosphonooxy)methyl]-imidazolidine-2,4-dione disodium salt in a single operation, a phosphoric acid diester 2,5-dioxo-4,4-diphenylimidazolidin-1-ylmethyl ester, whose phosphoric acid diester structural element can be selectively cleaved, is converted to 5,5-diphenyl-3-[(phosphonooxy)methyl]imidazolidine-2,4-dione and the latter is converted to its disodium salt; this feature—even taken on its own—is an essential part of the present invention.

The Examples which follow are intended to illustrate the invention in greater detail, but without in any way restricting its scope.

### EXAMPLE 1

a) Phosphoric acid 2,5-dioxo-4,4-diphenylimidazolidin-1-ylmethyl dibenzyl ester 28.2 g (100 mmol) of 3-hydroxymethyl-5,5-diphenylimidazolidine-2,4-dione (hydroxymethylphenytoin) were dissolved in 40 ml of acetonitrile. 31.6 g (400 mmol) of pyridine and 28.3 g (110 mmol) of dibenzyl phosphite in 15 ml of acetonitrile were added at 0-5° C. 17.6 g (110 mmol) of bromine were added slowly to the resulting solution. The solution turned brown towards the end of the addition. 20 g of water and 1 g of 36% sodium thiosulfate solution were added to the resulting reaction mixture, decolourization being rapidly observed. After the addition of a mixture of 40 ml of water and 40 ml of acetonitrile, the product crystallized out slowly at 0° C. The crystallization was completed by the addition of a further 20 ml of water. The product was filtered off and rinsed with a 1:1 mixture of cold acetonitrile and water; yield 30 g (55%).

b) Phosphoric acid 2,5-dioxo-4,4-diphenylimidazolidin-1-ylmethyl dibenzyl ester

155 g (550 mmol) of 3-hydroxymethyl-5,5-diphenylimidazolidine-2,4-dione (hydroxymethylphenytoin) were dissolved in 550 ml of acetonitrile. 130 g (1.3 mol) of pyridine were added at 0-5° C. A total of 134 g (750 mmol) of N-bromo-succinimide (NBS) were added to the resulting solution in four 33.5 g portions, and 200 g of dibenzyl phosphite (658.25 mmol, 87%) in 260 ml of acetonitrile were added in parallel over 6 h. 250 g of water and 50 g of 36% sodium thiosulfate solution (approx. 2 mmol/g) were added to the resulting orange suspension, decolourization being rapidly observed. After the addition of 650 ml of ethyl acetate, the phases were separated and the organic phase was washed with 50 g of saturated sodium carbonate solution. The organic phase was concentrated and the resulting yellow-orange oil was taken up in a mixture 350 ml of acetonitrile and 350 ml of water. Seeding produced a bulky precipitate, which was filtered off and dried under vacuum; yield 200 g (68%).

c) Phosphoric acid 2,5-dioxo-4,4-diphenylimidazolidin-1-ylmethyl diethyl ester

14.1 g (50 mmol) of 3-hydroxymethyl-5,5-diphenylimidazolidine-2,4-dione (hydroxymethylphenytoin) were dissolved in 25 ml of acetonitrile. 9.9 g (125 mol) of pyridine were added at 0-5° C. A total of 10.8 g (60 mmol) of N-bromo-succinimide (NBS) were added to the resulting solution in two 5.4 g portions, and 8.3 g (60 mmol) of diethyl phosphite in 15 ml of acetonitrile were added in parallel over 3 h. 20 g of water and 1 g of 36% sodium thiosulfate solution were added to the resulting orange suspension, decolourization being rapidly observed. After the addition of 50 ml of ethyl acetate, the phases were separated and the organic phase was washed with twice 20 ml of saturated sodium hydrogen carbonate solution and twice 20 ml of saturated sodium chloride solution. The organic phase was concentrated and the resulting yellow-orange oil was crystallized from a mixture of 20 ml of acetone and 20 ml of water. The precipitate was filtered off and dried under vacuum; yield 11.5 g (55%).

d) Phosphoric acid 2,5-dioxo-4,4-diphenylimidazolidin-1-ylmethyl diethyl ester

14.1 g (50 mmol) of 3-hydroxymethyl-5,5-diphenylimidazolidine-2,4-dione (hydroxymethylphenytoin) were dissolved in 25 ml of acetonitrile. 9.9 g (125 mol) of pyridine were added at 0-5° C. A total of 10.8 g (60 mmol) of N-bromo-succinimide (NBS) were added to the resulting solution in two 5.4 g portions, and 10.0 g (60 mmol) of triethyl phosphite in 15 ml of acetonitrile were added in parallel over 3 h. 20 g of water and 1 g of 36% sodium thiosulfate solution were added to the resulting orange suspension, decolourization being rapidly observed. After the addition of 50 ml of ethyl acetate, the phases were separated and the organic phase was washed with twice 20 ml of saturated sodium hydrogen carbonate solution and twice 20 ml of saturated sodium chloride solution. The organic phase was concentrated and the resulting yellow-orange oil was crystallized from a mixture of 20 ml of acetone and 20 ml of water. The precipitate was filtered off and dried under vacuum; yield 9.3 g (44%).

e) Phosphoric acid 2,5-dioxo-4,4-diphenylimidazolidin-1-ylmethyl diallyl ester

7.05 g (25 mmol) of 3-hydroxymethyl-5,5-diphenylimidazolidine-2,4-dione (hydroxymethylphenytoin) were dissolved in 15 ml of acetonitrile. 4.9 g (62 mol) of pyridine were added at 0-5° C. A total of 9.6 g (30 mmol) of carbon tetra-bromide were added to the resulting solution in three 3.2 g portions, and 6.1 g (30 mmol) of triallyl phosphite dissolved in 5.0 ml of acetonitrile were added in parallel over 2 h. 20 g of water were added to the resulting orange suspension. After the addition of 25 ml of ethyl acetate, the phases were separated and the organic phase was washed with twice 10 ml of saturated sodium hydrogen carbonate solution and twice 10 ml of saturated sodium chloride solution. The organic phase was concentrated and the resulting brownish oil was purified by column chromatography (solid phase: silica gel, eluent: petroleum ether/ethyl acetate 1:1-1:4). The resulting crude product was recrystallized once from isopropanol; yield 3.6 g (32%).

### EXAMPLE 2

5,5-Diphenyl3-[(phosphonooxy)methyl]imidazolidine-2,4-dione disodium salt

20 g (36.9 mmol) of phosphoric acid 2,5-dioxo-4,4-diphenylimidazolidin-1-ylmethyl dibenzyl ester were taken up in 100 ml of a 1 molar solution of sodium acetate in methanol (100 mmol, 2.7 eq) and 0.6 g (10 mmol) of glacial acetic acid. 200 ml of MeOH/water 80:20 and 1.5 g of palladium-on-active charcoal were added. The reaction mixture was hydrogenated for approx. 2.5 h at 40° C. and a pressure of 1.5-2.5 bar, during which part of the intermediate (monobenzyl ester Na salt) transiently precipitated out, although the stirrability of the reaction mixture remained good. It was filtered warm over Célite and concentrated on a rotary evaporator at a bath temperature of 45° C. The residue was diluted with 60 ml of a 1:1 water/MeOH mixture. The pH was adjusted to 8.5 by adding 34.2 g (48 mmol) of 15% Na₂CO₃ solution. The product began to precipitate out at pH 6.5. 20 ml of MeOH were added and the reaction mixture was cooled slowly to 0° C. and filtered with suction and the material on the filter was rinsed with MeOH/water 1:1; moist weight 33.3 g. The crude product was recrystallized from 66 ml of a 1:1 acetone/water mixture, rinsed with cold acetone/water and dried under vacuum; yield 18.8 g, with 22.9% water content 35.7 mmol, 97%.

1. Process for the preparation of 5,5-diphenyl-3-[(phosphonooxy)methyl]imidazolidine-2,4-dione disodium salt, characterized in that 3-hydroxymethyl-5,5-diphenylimidazoline-2,4-dione is reacted with a phosphorous acid diester or triester activated by an oxidizing agent, whose ester groups can be selectively cleaved from the reaction product, the ester groups are cleaved from the resulting phosphoric acid diester 2,5-dioxo-4,4-diphenylimidazolidin-1-ylmethyl ester and the resulting 5,5-diphenyl-3-[(phosphonooxy)methyl]imidazolidine-2,4-dione is converted to its disodium salt.

2. Process according to claim 1, characterized in that the oxidizing agent is a halogenating agent.

3. Process according to claim 2, characterized in that the halogenating agent is elemental bromine, N-bromosuccinimide, 1,3-dibromo-5,5-dimethylhydantoin, carbon tetrabromide, trichlorobromomethane, elemental chlorine, N-chloro-succinimide, trichloroisocyanuric acid or hexachloroacetone.

4. Process according to claim 1, characterized in that the ester groups can be cleaved under mild acidic conditions, oxidatively, photochemically, under mild basic conditions or by hydrogenolysis.

5. Process according to claim 4, characterized in that the phosphorous acid ester is ditert-butyl phosphite, dibenzyl phosphite, bis-4-methoxybenzyl phosphite, bis-4-bromobenzyl phosphite, bis-4-nitrobenzyl phosphite, bis(2,4-dimethoxybenzyl) phosphite, bis-2,2,2-trichloroethyl phosphite, bis(2-trimethylsilylethyl) phosphite, triallyl phosphite or tribenzyl phosphite, or dimethyl phosphite, diethyl phosphite, trimethyl phosphite or triethyl phosphite.

6. Process according to claim 1, characterized in that 3-hydroxymethyl-5,5-diphenylimidazoline-2,4-dione is reacted with dibenzyl phosphite activated by elemental bromine or N-bromosuccinimide, the resulting phosphoric acid 2,5-dioxo-4,4-diphenylimidazolidin-1-ylmethyl dibenzyl ester is hydrogenated and the resulting 5,5-diphenyl-3-[(phosphonooxy)methyl]imidazolidine-2,4-dione is converted to its disodium salt with sodium carbonate.

7. Process according to claim 6, characterized in that the hydrogenation takes place in the presence of palladium-on-active charcoal.

8. Process according to claim 6, characterized in that on the one hand the activation of the dibenzyl phosphite with elemental bromine or N-bromosuccinimide and the reaction of the activation product with 3-hydroxymethyl-5,5-diphenylimidazoline-2,4-dione, and on the other hand the hydrogenation of the phosphoric acid 2,5-dioxo-4,4-diphenylimidazolidin-1-ylmethyl dibenzyl ester and the conversion of the 5,5-diphenyl-3-[(phosphonooxy)methyl]imidazolidine-2,4-dione to its disodium salt, are each carried out in one operation.

9. Process according to claim 8, characterized in that the activation of the dibenzyl phosphite with elemental bromine or N-bromosuccinimide and the reaction of the activation product with 3-hydroxymethyl-5,5-diphenylimidazoline-2,4-dione are carried out in a mixture of acetonitrile and pyridine, and the hydrogenation of the phosphoric acid 2,5-dioxo-4,4-diphenylimidazolidin-1-ylmethyl dibenzyl ester and the conversion of the 5,5-diphenyl-3-[(phosphonooxy)-methyl]imidazolidine-2,4-dione to its disodium salt are carried out in a mixture of methanol, water and an acetic acid/sodium acetate buffer.

10. Process for the preparation of 5,5-diphenyl-3-[(phosphonooxy)methyl]imidazolidine-2,4-dione disodium salt, characterized in that, in a single operation, a phosphoric acid diester 2,5-dioxo-4,4-diphenylimidazolidin-1-ylmethyl ester, whose phosphoric acid diester structural element can be selectively cleaved, is converted to 5,5-diphenyl-3-[(phosphonooxy)-methyl]imidazolidine-2,4-dione and the latter is converted to its disodium salt.

11. Process according to claim 2, characterized in that the ester groups can be cleaved under mild acidic conditions, oxidatively, photochemically, under mild basic conditions or by hydrogenolysis.

12. Process according to claim 3, characterized in that the ester groups can be cleaved under mild acidic conditions, oxidatively, photochemically, under mild basic conditions or by hydrogenolysis.

13. Process according to claim 7, characterized in that on the one hand the activation of the dibenzyl phosphite with elemental bromine or N-bromosuccinimide and the reaction of the activation product with 3-hydroxymethyl-5,5-diphenylimidazoline-2,4-dione, and on the other hand the hydrogenation of the phosphoric acid 2,5-dioxo-4,4-diphenylimidazolidin-1-ylmethyl dibenzyl ester and the conversion of the 5,5-diphenyl-3-[(phosphonooxy)methyl]imidazolidine-2,4-dione to its disodium salt, are each carried out in one operation.
